(19) United States
(12) Patent Application Publication
(10) Pub. No.: US 2025/0134018 A1
(43) Pub. Date: May 1, 2025

(54) METHOD FOR INDUCING IRIS DOMESTICA SEED BUDS TO GENERATE POLYPLOID

(71) Applicant: Yangtze Normal University, Chongqing (CN)

(72) Inventors: Wenji Xu, Chongqing (CN); Yuhan Fang, Chongqing (CN); Gangjun Luo, Chongqing (CN); Yihong Xiang, Chongqing (CN); Xuemei Zhang, Chongqing (CN); Jie Luo, Chongqing (CN); Guochao Hu, Chongqing (CN); Yongyao Fu, Chongqing (CN)

(21) Appl. No.: 18/642,636

(22) Filed: Apr. 22, 2024

(30) Foreign Application Priority Data
Oct. 25, 2023 (CN) 202311392075.4

(51) Int. Cl. A01H 1/08

(52) U.S. Cl. CPC A01H 1/08

(57) ABSTRACT

Disclosed is a method for inducing Iris domestica seed buds to generate polyploid, including the following steps: seed bud induction: inducing Iris domestica seeds at a low temperature, then soaking, sterilizing and germinating the same to obtain Iris domestica seed buds; and polyploid induction: soaking the obtained Iris domestica seed buds in oryzalin solution for polyploid induction to obtain mutagenized buds. In the present disclosure, the Iris domestica seed buds are treated by soaking with oryzalin to induce polyploid, and a total of 737 new germplasms of polyploid Iris domestica are obtained. The breeding has a short experimental period, simple operations, and high polyploid induction efficiency. At the same time, polyploids with large flowers, bright colors and strong plants are obtained. The method lays the foundations for the polyploid breeding of Iris domestica and the cultivation of new varieties of Iris domestica with large flower type and strong resistance.

### CROSS-REFERENCE TO RELATED APPLICATIONS

This application claims priority of Chinese Patent Application No. 202311392075. 4, filed on Oct. 25, 2023, the entire contents of which are incorporated herein by reference.

### TECHNICAL FIELD

The present disclosure relates to the technical field of plant polyploid breeding, in particular to a method for inducing Iris domestica seed buds to generate polyploid.

### BACKGROUND

Iris domestica (2n=2x=32), a perennial herb of Iris in Iridaceae, has a flowering period from May to August and orange flowers, which is rare in Iris and has certain ornamental value; and has the characteristics of cold tolerance, drought tolerance, barren tolerance and long flowering period. Iris domestica is one of the important ornamental flowers in landscaping and can also be used as medicinal plants. At present, the planted Iris domestica is diploid, with small flowers, which needs further improvement. Polyploid plants have the characteristics of strong growth, bright flowers, strong resistance and high yield. Therefore, polyploid breeding is an important means to cultivate new varieties of ornamental flowers, which has been widely developed for some flowers, such as Lilium brownii, Chrysanthemum, Zantedeschia aethiopica, Dianthus caryophyllus and Cyclamen persicum. However, there is no research report on polyploid breeding of Iris domestica.

Previously, flower polyploid breeding is performed under tissue culture conditions, taking bulbs, protocorms, somatic embryos and sterile buds as materials, by using culture medium method and co-culture method for induction, which has the problem of easy contamination.

### SUMMARY

In order to solve the above technical problems, the present disclosure provides a method for inducing Iris domestica seed buds to generate polyploid.

A method for inducing Iris domestica seed buds to generate polyploid includes the following steps:
(1) seed bud induction: inducing Iris domestica seeds at a low temperature, then soaking, sterilizing and germinating the same to obtain Iris domestica seed buds; and
(2) polyploid induction: soaking the Iris domestica seed buds obtained in step 1 in oryzalin solution with a mass fraction of 0.03-0.07 wt % for 18-36 h for polyploid induction to obtain mutagenized buds.

Preferably, a low induction temperature in step 1 is 4-6° C.

Preferably, a low temperature induction time in step 1 is 30-40 d.

Preferably, the Iris domestica seeds are soaked with water for 30-36 h.

Preferably, in step 1, 1.5%-2.0% sodium hypochlorite solution is used for soak and sterilization for 20-30 min.

Preferably, the germination is performed for 10-20 d.

Preferably, in step 2, a length of the Iris domestica seed bud is 0.5-1 mm.

Preferably, in step 2, the polyploid induction is performed by shaking.

Preferably, the method further includes the following steps: transplanting mutagenized seedlings, moisturizing the mutagenized buds obtained in step 2, and performing illumination culture, the illumination culture conditions including a temperature of 23-25° C., a daytime illumination of 2000 lux for 14 h, and darkness at night for 10 h; and
transferring the seed buds into a nutrition pot after 2-3 cotyledons grow out, and putting the seedlings into a greenhouse for cultivation for 50-60 d after transplanting.

Preferably, the substrate of the nutrition pot is peat soil from Shandong mandelai agriculture technology co., ltd.

Compared with the related art, the present disclosure has the following beneficial effects.

1. In the present disclosure, the Iris domestica seed buds are treated with oryzalin solution to generate polyploid. The polyploid Iris domestica is embodied in that the flowers become bigger, the flower color is brilliant, and the plants become stronger, etc. Therefore, compared with diploid Iris domestica, the polyploid Iris domestica has higher ornamental and economic value, and has greater development potential.

2. In the present disclosure, the method of inducing polyploid of Iris domestica with oryzalin has the advantages of simple operations, short treatment periods, high induction rates and few chimeras. The treatment of 0.50% oryzalin for 24 h has the best effect on inducing the polyploid of Iris domestica, an induction rate being as high as 44.00%, and 132 polyploid plants are obtained.

### BRIEF DESCRIPTION OF THE DRAWINGS

FIG. 1 is a photograph showing the growth of Iris domestica seed buds after being inducted by oryzalin, where, a shows the growth of a control group for 7 d, b shows the growth of the Iris domestica seed buds treated by 0.05 wt % oryzalin for 12 h for 7 d, and c shows the growth of the Iris domestica seed buds treated by 0.05 wt % oryzalin for 12 h for 14 d;

FIG. 2 is a picture of a flow cytometer (FCM) of polyploid Iris domestica, where a is diploid, and b-i are polyploidies 3-54, 3-59, 3-110, 3-186, 3-267, 3-293, 3-441 and 3-446, respectively;

FIG. 3 is a picture of chromosomes of polyploid Iris domestica, where a is diploid, and b-d are polyploidies 3-59, 3-419 and 3-427, respectively;

FIG. 4 is a picture of flower morphology of polyploid Iris domestica, where a is diploid, and b-i are polyploidies 3-186, 3-226, 3-267, 3-271, 3-325, 3-337, 3-419 and 3-446, respectively; and

FIG. 5 is a picture of pollen morphology of polyploid Iris domestica, where a is diploid, and b-i are polyploidies 3-45, 3-54, 3-110, 3-115, 3-186, 3-229, 3-271, 3-337, respectively.

### DETAILED DESCRIPTION

Hereinafter, specific examples of the present disclosure will be described in detail, but it is to be understood that the scope of protection of the present disclosure is not limited by the specific examples. Based on the examples in the present disclosure, all other examples obtained by those of ordinary skill in the art without creative efforts belong to the scope of protection of the present disclosure. Unless otherwise specified, the experimental methods described in each example of the present disclosure are all conventional methods.

In the present disclosure, the oryzalin solution is prepared by dissolving oryzalin with a small amount of methanol, an amount of methanol used in the present disclosure being 2-3 mL. The oryzalin solution with a concentration of 0.03%-0.09 wt % is prepared using sterile water, and then filtered through a 0.22 μm sterile microporous filter membrane.

In the present disclosure, a high parental rate refers to a ratio higher than diploid.

### Example 1

A method for inducing Iris domestica seed buds to generate polyploid includes the following steps.

At (1): seed bud induction
normally saturated Iris domestica seeds were selected, filled into sealed ziplock bags and placed at 4° C. for low temperature storage induction for 30 d. The Iris domestica seeds inducted at the low temperature were taken out and healthy seeds were selected and washed with water for 15 min and soaked for 18 h, then soaked and sterilized with 1.5% sodium hypochlorite solution for 20 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 10 d. The 30/20° C. illumination refers to that daytime illumination is at 2000 lux for 12 h at a temperature of 30° C., and the darkness at night lasts for 12 h at a temperature of 20° C.

At (2): polyploid induction
the Iris domestica seed buds obtained in step (1), having a bud length of about 0.5 mm, were soaked in oryzalin solution with a mass fraction of 0.03 wt % for polyploid induction, under the condition of protection from light, and placed on a shaking table for 18 h with a shaking speed of 60 g/min; and the seed buds were taken out after being inducted, washed with sterile water twice, and then soaked in the sterile water for 1 h.

### Example 2

A method for inducing Iris domestica seed buds to generate polyploid includes the following steps.

At (1): seed bud induction
normally saturated Iris domestica seeds were selected, filled into sealed ziplock bags and placed at 5° C. for low temperature storage induction for 35 d. The Iris domestica seeds inducted at the low temperature were taken out and healthy seeds were selected and washed with water for 20 min and soaked for 24 h, then soaked and sterilized with 2.0% sodium hypochlorite solution for 20 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 20 d. The 30/20° C. illumination refers to that daytime illumination is at 2000 lux for 12 h at a temperature of 30° C.; and the darkness at night lasts for 12 h at a temperature of 20° C.

At (2): polyploid induction

the Iris domestica seed buds obtained in step (1), having a bud length of about 1 mm, were soaked in oryzalin solution with a mass fraction of 0.05 wt % for polyploid induction, under the condition of protection from light, and placed on a shaking table for 36 h with a shaking speed of 60 g/min; and the seed buds were taken out after being inducted, washed with sterile water for 3 times, and then soaked in the sterile water for 2 h.

### Example 3

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.07 wt % for polyploid induction, under the condition of protection from light, and placed on a shaking table for 24 h with a shaking speed of 60 g/min; and the seed buds were taken out after being inducted, washed with sterile water twice, and then soaked in the sterile water for 2 h.

### Example 4

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.03 wt % for polyploid induction, under the condition of protection from light, and placed on a shaking table for 30 h with a shaking speed of 60 g/min; and the seed buds were taken out after being inducted, washed with sterile water twice, and then soaked in the sterile water for 2 h.

### Example 5

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.03 wt % for polyploid induction, under the condition of protection from light, and placed on a shaking table for 36 h with a shaking speed of 60 g/min; and the seed buds were taken out after being inducted, washed with sterile water twice, and then soaked in the sterile water for 2 h.

### Example 6

A method for inducing Iris domestica seed buds to generate polyploid includes the following steps.

At (1): seed bud induction normally saturated Iris domestica seeds were selected, filled into sealed

ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.05 wt % for polyploid induction, under the condition of protection from light, and placed on a shaking table for 24 h with a shaking speed of 60 g/min; and the seed buds were taken out after being inducted, washed with sterile water twice, and then soaked in the sterile water for 2 h.

### Example 7

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.05 wt % for polyploid induction, under the condition of protection from light, and placed on a shaking table for 18 h with a shaking speed of 60 g/min; and the seed buds were taken out after being inducted, washed with sterile water twice, and then soaked in the sterile water for 2 h.

### Example 8

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2) polyploid induction the Iris domestica seed buds obtained in step (1), having a bud length of 0.8 mm, were soaked in oryzalin solution with a mass fraction of 0.07 wt % for polyploid induction, under the condition of protection from light, and placed on a shaking table for 18 h with a shaking speed of 60 g/min; and the seed buds were taken out after being inducted, washed with sterile water twice, and then soaked in the sterile water for 2 h.

### Example 9

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.07 wt % for polyploid induction, under the condition of protection from light, and placed on a shaking table for 36 h with a shaking speed of 60 g/min; and the seed buds were taken out after being inducted, washed with sterile water twice, and then soaked in the sterile water for 2 h.

### Example 10

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.03 wt % for polyploid induction, under the condition of protection from light, and placed on a shaking table for 24 h with a shaking speed of 60 g/min; and the seed buds were taken out after being inducted, washed with sterile water twice, and then soaked in the sterile water for 2 h.

### Example 11

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.05 wt % for polyploid induction, under the condition of protection from light, and placed on a shaking table for 18 h with a shaking speed of 60 g/min; and the seed buds were taken out after being inducted, washed with sterile water twice, and then soaked in the sterile water for 2 h.

### Example 12

A method for inducing Iris domestica seed buds to generate polyploid includes the following steps.

At (1): seed bud induction normally saturated Iris domestica seeds were selected, filled into sealed ziplock bags and placed at 6° C. for low temperature storage induction for 40 d. The Iris domestica seeds inducted at the low temperature were taken out and healthy seeds were selected and washed with water for 18 min and soaked for 36 h, then soaked and sterilized with 2.0% sodium hypochlorite solution for 30 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of 0.8 mm, were soaked in oryzalin solution with a mass fraction of 0.07 wt % for polyploid induction, under the condition of protection from light, and placed on a shaking table for 30 h with a shaking speed of 60 g/min; and the seed buds were taken out after being inducted, washed with sterile water twice, and then soaked in the sterile water for 2 h.

### Comparative Example 1

A method for inducing Iris domestica seed buds to generate polyploid includes the following steps.

At (1): seed bud induction normally saturated Iris domestica seeds were selected, filled into sealed ziplock bags and placed at 4° C. for low temperature storage induction for 30 d. The Iris domestica seeds inducted at the low temperature were taken out and healthy seeds were selected and washed with water for 18 min and soaked for 36 h, then soaked and sterilized with 2.0% sodium hypochlorite solution for 20 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of about 1 mm, were soaked in oryzalin solution with a mass fraction of 0.05 wt % for polyploid induction, under the condition of protection from light, and placed on a shaking table for 48 h with a shaking speed of 60 g/min; and the seed buds were taken out after being inducted, washed with sterile water for 2-3 times, and then soaked in the sterile water for 2 h.

### Comparative Example 2

A method for inducing Iris domestica seed buds to generate polyploid includes the following steps.

At (1): seed bud induction normally saturated Iris domestica seeds were selected, filled into sealed ziplock bags and placed at 4° C. for low temperature storage induction for 30 d. The Iris domestica seeds inducted at the low temperature were taken out and healthy seeds were selected and washed with water for 18 min and soaked for 36 h, then soaked and sterilized with 2.0% sodium hypochlorite solution for 20 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of about 0.5 mm, were soaked in oryzalin solution with a mass fraction of 0.09 wt % for polyploid induction, under the condition of protection from light, and placed on a shaking table for 24 h with a shaking speed of 60 g/min; and the seed buds were taken out after being inducted, washed with sterile water for 3 times, and then soaked in the sterile water for 2 h.

### Comparative Example 3

A method for inducing Iris domestica seed buds to generate polyploid includes the following steps.

At (1): seed bud induction normally saturated Iris domestica seeds were selected, filled into sealed ziplock bags and placed at 4° C. for low temperature storage induction for 30 d. The Iris domestica seeds inducted at the low temperature were taken out and healthy seeds were selected and washed with water for 18 min and soaked for 36 h, then soaked and sterilized with 2.0% sodium hypochlorite solution for 20 min and washed with sterile water twice, and the surface moisture of seeds was dried. A clean plastic tray was prepared, wet gauzes were spread, and the sterilized seeds were spread, sealed with preservative films, and putted into a 30/20° C. illumination incubator for germination for 15 d. The 30/20° C. illumination refers to that daytime illumination is at 2000 lux for 12 h at a temperature of 30° C.; and the darkness at night lasts for 12 h at a temperature of 20° C.

At (2): polyploid induction the Iris domestica seed buds obtained in step (1), having a bud length of about 1 mm, were soaked in oryzalin solution with a mass fraction of 0.09 wt % for polyploid induction, under the condition of protection from light, and placed on a shaking table for 36 h with a shaking speed of 60 g/min; and the seed buds were taken out after being inducted, washed with sterile water for 3 times, and then soaked in the sterile water for 2 h.

### Comparative Example 4

Comparative Example 4 differed from Comparative Example 1 in that the Iris domestica seed buds were soaked in oryzalin solution with a mass fraction of 0.03 wt % for polyploid induction, under the condition of protection from light, and placed on a shaking table for 48 h, and the rest conditions and steps were the same as those in Comparative Example 1.

### Comparative Example 5

Comparative Example 5 differed from Comparative Example 1 in that the Iris domestica seed buds were soaked in oryzalin solution with a mass fraction of 0.07 wt % for polyploid induction, under the condition of protection from light, and placed on a shaking table for 48 h, and the rest conditions and steps were the same as those in Comparative Example 1.

### Comparative Example 6

Comparative Example 6 differed from Comparative Example 2 in that the Iris domestica seed buds were soaked in oryzalin solution with a mass fraction of 0.09 wt % for polyploid induction, under the condition of protection from light, and placed on a shaking table for 18 h, and the rest conditions and steps were the same as those in Comparative Example 1.

### Comparative Example 7

Comparative Example 7 differed from Comparative Example 2 in that the Iris domestica seed buds were soaked in oryzalin solution with a mass fraction of 0.09 wt % for polyploid induction, under the condition of protection from light, and placed on a shaking table for 30 h, and the rest conditions and steps were the same as those in Comparative Example 1.

### Comparative Example 8

Comparative Example 8 differed from Comparative Example 2 in that the Iris domestica seed buds were soaked in oryzalin solution with a mass fraction of 0.09 wt % for polyploid induction, under the condition of protection from light, and placed on a shaking table for 48 h, and the rest conditions and steps were the same as those in Comparative Example 1.

The mutagenized buds obtained in various examples and comparative examples were subjected to the following treatments.

(1) Mutagenized seedling transplanting

The plastic tray was spread with wet gauzes, and the mutagenized buds induced in step (2) were spread on the gauzes for cultivation, sealed with preservative films for moisturizing, and put into an illumination culture chamber for cultivation at a temperature of 24° C. under a daytime illumination of 2000 lux for 14 h, and under darkness at night for 10 h. When two cotyledons of the seed buds grew out, the seed buds were transferred into a 10×10 cm nutrition pot, the substrate was peat soil from Shandong mandelai agriculture technology co., ltd., and after transplanting, the seedlings were put into a greenhouse for cultivation for 50 d to obtain the mutagenized seedlings. It is to be noted that when the seedlings are transferred into the nutrition pot, the bud grows 2-3 cotyledons, which can achieve similar results. In the experiments of the present disclosure, in order to unify, 2 cotyledons are selected, and after transplanting, the seedlings are put into the greenhouse for cultivation for 50-60 d, which can achieve similar results. In the experiments of the present disclosure, in order to unify the time for each group, 50 d is selected.

### (2) Mutagenized Seedling Ploidy Identification

The mutagenized seedlings were identified, and the top tender green leaves of the plant were selected for chromosome ploidy identification by the FCM. The chromosome numbers of root tips with vigorous division were observed by chromosome slide method.

FCM identification procedures were as follows: 0.5 g of top tender green leaves of the mutagenic seedlings were taken and put into a glass dish pre-cooled at 4° C., and then put on an ice box with the addition of 2 mL of cell extract; the leaves were quickly cut into homogenate by using a blade, then filtered to a 2 mL centrifuge tube using a flow filter to obtain a cell solution, and shaded and put into a refrigerator at 4° C. for 10 min; and 500 μL of cell solution was taken into a flow tube, with the addition of 40 μL of PI dye solution, then put into an ice box to stain 30 min protected from light, and finally detected on an ACEA FCM.

Observation procedures for chromosome number of the root tips were as follows: the root tips with vigorous division were taken at 9:00 a. m.-12:00 a. m., the substrate and soil were cleaned with clean water, soaked with 0.002 mol/L 8-hydroxyquinoline solution for 4 h, fixed with Carnoy's fluid for 4 h, transferred to 75% alcohol, and stored in a refrigerator at 4° C. The stored root tips were taken out, dissociated 9 min with 5 mol/L hydrochloric acid and washed with water, the root tip meristem was cut and placed on a glass slide, 3 drops of magenta dye solution were added, staining for 0.5 h, and finally the situation was observed and photos were taken under the microscope. It is to be noted that the dyeing method of this section is exemplary to use a specific dyeing method, and other dyeing methods may be substituted, which are all within the inventive concept of the present disclosure.

### (3) Field Planting of Polyploid Plants

A flat experimental field was selected, and plowed twice with a tractor with a depth of 35 cm, 2 tons of organic fertilizer and 10 kilograms of compound fertilizer per mu were applied with a content ratio of nitrogen, phosphorus to potassium of 15-15-15, and spread evenly in the field, and the identified polyploid seedlings were planted with the row spacing of 40×50 cm.

(4) The morphology of polyploid plants was observed, and the plant height, leaf length, leaf width, leaf morphology, flower diameter, lengths and widths of inner and outer perianths of polyploid plants were investigated.

(5) Pollen sizes of polyploid plants were observed by taking out the anthers in the morning, dispersing a same in a sulfuric acid paper box, and then air-drying the same in a dry and cool place, and scattering the collected pollen on a glass slide, and finally observing the pollen sizes using an optical microscope (Olympus).

The effect of oryzalin on the germination of Iris domestica seed buds was studied, and the results were shown in Table 1.

TABLE 1
Germination rate and survival rate of Iris domestica seed buds treated by oryzalin

| Treatment | | | | | |
|---|---|---|---|---|---|
| concentration | | | | | |
| of | Treatment | Total number of | Germination | Survival | |
| oryzalin/wt % | time/h | treatments/pellet | rate | rate | Treatment |
| 0.03% | 18 | 300 | 100.00% | 60.33% | Example 1 |
| | 24 | 300 | 100.00% | 26.00% | Example 10 |
| | 30 | 300 | 100.00% | 49.33% | Example 4 |
| | 36 | 300 | 91.67% | 58.91% | Example 5 |
| | 48 | 300 | 29.67% | 16.00% | Comparative |
| | | | | | Example 4 |
| 0.05% | 18 | 300 | 83.00% | 53.41% | Example 11 |
| | 24 | 300 | 96.00% | 87.50% | Example 6 |
| | 30 | 300 | 90.00% | 31.11% | Example 7 |
| | 36 | 300 | 90.00% | 25.93% | Example 2 |
| | 48 | 300 | 25.33% | 10.33% | Comparative |
| | | | | | Example 1 |
| 0.07% | 18 | 300 | 96.33% | 38.75% | Example 8 |
| | 24 | 300 | 77.33% | 38.79% | Example 3 |
| | 30 | 300 | 93.33% | 23.57% | Example 12 |
| | 36 | 300 | 68.00% | 43.63% | Example 9 |
| | 48 | 300 | 14.00% | 5.33% | Comparative |
| | | | | | Example 5 |
| 0.09% | 18 | 300 | 88.67% | 13.16% | Comparative |
| | | | | | Example 6 |
| | 24 | 300 | 68.00% | 3.43% | Comparative |
| | | | | | Example 2 |
| | 30 | 300 | 56.00% | 0.60% | Comparative |
| | | | | | Example 7 |
| | 36 | 300 | 44.00% | 0 | Comparative |
| | | | | | Example 3 |
| | 48 | 300 | 9.33% | 0 | Comparative |
| | | | | | Example 8 |

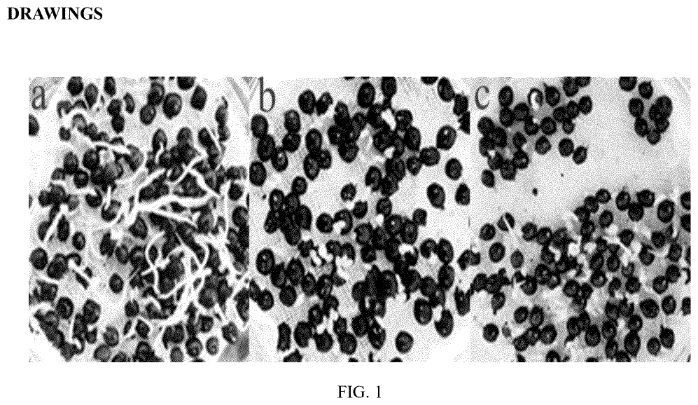

A total of 1508 mutagenized seedlings were obtained by treating the Iris domestica seed buds with oryzalin. After being treated with 0.03%, 0.05%, 0.07% and 0.09% oryzalin for 18-36 h, the germination rates of Iris domestica seed buds all exceeded 44.00%, as shown in FIG. 1. The lower the treatment concentration, the shorter the treatment time, the higher the germination rate. After being treated for 48 h, the germination rates were all lower than 30.00%. The analysis of the survival rate of Iris domestica seed buds showed that the higher the treatment concentration, the lower the survival rate, but there was no obvious regularity at 18 h, 24 h, 30 h and 36 h at the same treatment concentration. The highest survival rate was 87.50% at 0.05% treatment concentration for 24 h, the survival rate was lowest at 0.09% treatment concentration for 36 h and 48 h, and no mutagenized seedlings were obtained.

TABLE 2
Ploidy increasing rate of Iris domestica seed buds treated by oryzalin

| Treatment | | Total | Surviving | Polyploid | Ploidy | |
|---|---|---|---|---|---|---|
| concentration | Treatment | number of | plant/ | plant/ | increasing | |
| of oryzalin/wt % | time/h | treatments/pellet | strain | strain | rate | Treatment |
| 0.03% | 18 | 300 | 181 | 60 | 20.00% | Example 1 |
| | 24 | 300 | 78 | 9 | 3.00% | Example 10 |
| | 30 | 300 | 148 | 56 | 18.67% | Example 4 |
| | 36 | 300 | 162 | 61 | 20.33% | Example 5 |
| | 48 | 300 | 48 | 7 | 2.33% | Comparative |
| | | | | | | Example 4 |
| 0.05% | 18 | 300 | 133 | 75 | 25.00% | Example 11 |
| | 24 | 300 | 252 | 132 | 44.00% | Example 6 |
| | 30 | 300 | 84 | 53 | 17.67% | Example 7 |
| | 36 | 300 | 70 | 44 | 14.67% | Example 2 |
| | 48 | 300 | 31 | 8 | 2.67% | Comparative |
| | | | | | | Example 1 |
| 0.07% | 18 | 300 | 112 | 66 | 22.00% | Example 8 |
| | 24 | 300 | 90 | 53 | 17.67% | Example 3 |
| | 30 | 300 | 66 | 41 | 13.67% | Example 12 |
| | 36 | 300 | 89 | 46 | 15.33% | Example 9 |
| | 48 | 300 | 16 | 4 | 1.33% | Comparative |
| | | | | | | Example 5 |
| 0.09% | 18 | 300 | 35 | 21 | 7.00% | Comparative |
| | | | | | | Example 6 |
| | 24 | 300 | 7 | 1 | 0.33% | Comparative |
| | | | | | | Example 2 |
| | 30 | 300 | 1 | 0 | 0 | Comparative |
| | | | | | | Example 7 |
| | 36 | 300 | 0 | 0 | 0 | Comparative |
| | | | | | | Example 3 |
| | 48 | 300 | 0 | 0 | 0 | Comparative |
| | | | | | | Example 8 |

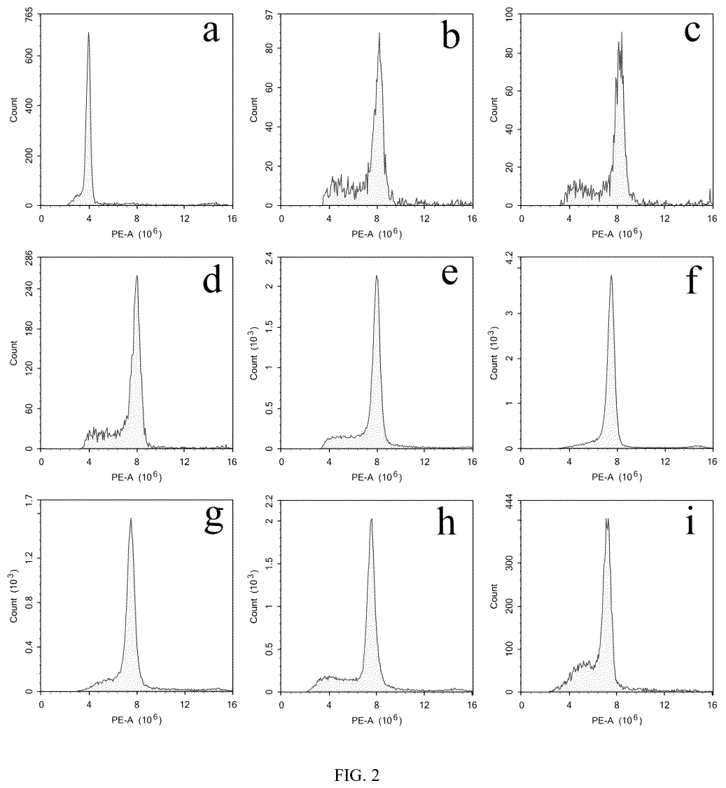

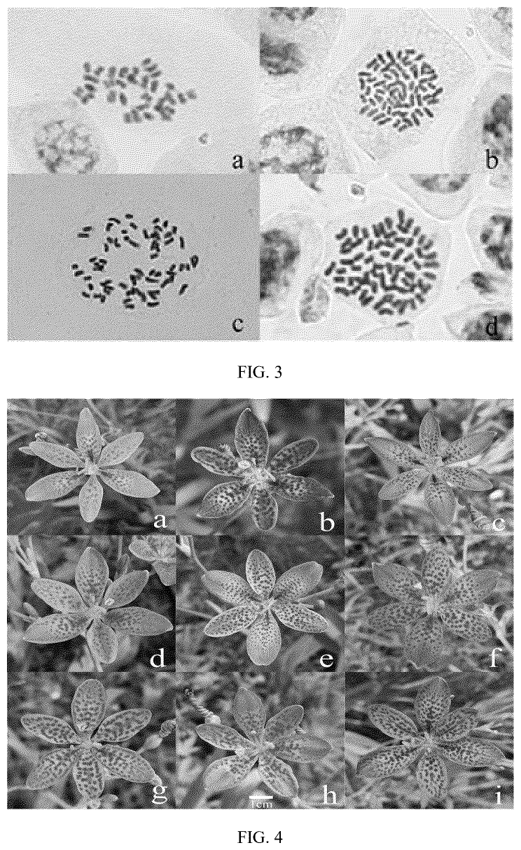

It could be seen from Table 2 that the best effect appeared when the polyploid of Iris domestica was induced by 0.05% oryzalin, followed by 0.03%, 0.07% and 0.09%. The ploidy increasing effect of 0.05% oryzalin treatment for 24 h was best, and the ploidy increasing rate was as high as 44.00%. The ploidy increasing rates of 0.03% concentration treatment for 18 h and 36 h, and 0.05% and 0.07% concentration treatments for 18 h were more than 20%; however, the ploidy increasing rate of 0.09% concentration treatment for 30 h, 36 h and 48 h was the lowest, which was 0. When the treatment times were 18 h and 24 h, the ploidy increasing rate increased first and then decreased with the increase of the concentration of oryzalin. When the treatment times were 30 h and 36 h, the ploidy increasing rate showed a downward trend with the increase of the concentration of oryzalin. As shown in FIG. 2, an abscissa is a nuclear fluorescence intensity and an ordinate is the number of nucleus to determine whether the plant is polyploid, for example, if a control diploid fluorescence intensity is 4, then a tetraploid fluorescence intensity is to be around 8. The results showed that a total of 737 polyploids were obtained in the experiments through FCM identification, as shown in FIGS. 2 and 3, including polyploids numbered 3-54, 3-59, 3-110, 3-186, 3-267, 3-293, 3-441, 3-446, 3-419, 3-427, 3-226, 3-271, 3-325, 3-337, 3-45, 3-115 and 3-229.

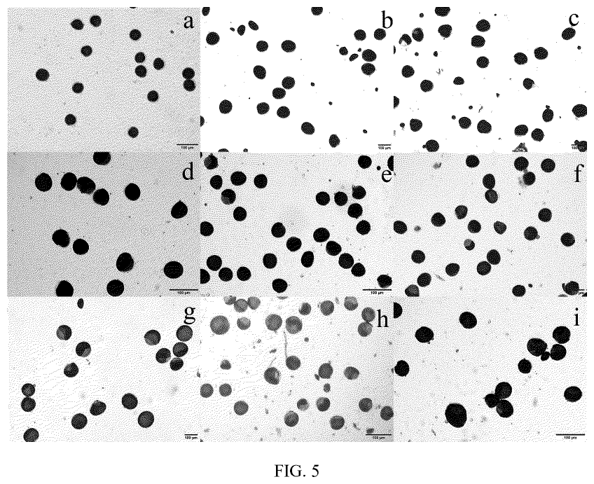

The morphological characters of 50 polyploid Iris domesticas were analyzed, including the plant height, leaf length, leaf width, leaf morphology, flower diameter, lengths and widths of inner and outer perianths. The results showed that stems and leaves of some polyploid Iris domestica plants became curved, the plants became more robust, the flower color changed, orange-red color and deep orange-red color appeared, the spot color became deeper, and flower spots of some polyploid plants increased significantly, as shown in FIG. 4. The plant heights of polyploid plants ranged from 66.0 cm to 152.0 cm, most of plants were lower than diploid, and only 14.00% of plants were higher than diploid. An average leaf length was less than a leaf length of diploid, and an average leaf width was greater than a leaf width of diploid, 42.00% of plants having larger leaf width than diploid. An average flower diameter of polyploid plants was 5.91 cm, which was larger than that of diploid. The average flower diameter of diploid was 5.40 cm, and 52.00% of plants were higher than diploid. In the polyploid plants, an average length and width of outer perianth, and an average length and width of inner perianth were greater than these of diploid. A coefficient of variation (CV) of plant height of polyploid plants was the largest, which was 18.00%; and a CV of corolla breadth was the smallest, which was 9.14%, as shown in Table 3. The 11 strain lines ‘3-48’, ‘3-52’, ‘3-102’, ‘3-104’, ‘3-124’, ‘3-271’, ‘3-289’, ‘3-325’, ‘3-337’, ‘3-347’ and ‘3-446’ of polyploid Iris domesticas are found to have large flowers, bright colors and strong plants by morphological analysis, which have strong utilization value.

TABLE 3
Morphological analysis of polyploid Iris domestica

| | | | Coefficient of | | High parental |
|---|---|---|---|---|---|
| Morphological index | Diploid | Polyploid | variation | Scope | rate |
| Plant height (cm) | 130.0 | 114.7 | 18.00% | 66.0-152.0 | 14.00% |
| Leaf length (cm) | 45.5 | 41.3 | 13.75% | 32.7-53.0 | 18.00% |
| Leaf width (cm) | 3.70 | 3.81 | 17.59% | 2.75-5.35 | 42.00% |
| Corolla breadth (cm) | 5.40 | 5.91 | 9.14% | 4.40-7.20 | 52.00% |
| Outer perianth length (cm) | 2.90 | 3.06 | 10.78% | 2.30-3.70 | 32.00% |
| Outer perianth width (cm) | 1.20 | 1.29 | 15.50% | 0.80-2.10 | 38.00% |
| Inner perianth length (cm) | 2.50 | 2.74 | 9.49% | 2.20-3.30 | 42.00% |
| Inner perianth width (cm) | 1.00 | 1.12 | 16.07% | 0.85-1.80 | 54.00% |

Pollen sizes of 50 polyploid Iris domesticas were observed. Anthers were taken in the morning, dispersed in a sulfuric acid paper box, and then air-dried in a dry and cool place, and the collected pollens were scattered on a glass slide, and finally the pollen sizes were observed by using an optical microscope (Olympus).

The pollen morphology and size of polyploid Iris domestica had some variation. The pollen morphology: oval, round and irregular pollens appeared; and the pollen size: large, medium, small and wizened pollens appeared (as shown in FIG. 5). A pollen longitudinal diameter of polyploid Iris domestica ranged from 44.30-98.22 μm, with an average of 65.83 μm and a CV of 18.36%, and the ploidies of 54.00% of the plants were greater than that of diploid. A pollen transverse diameter of polyploid Iris domestica ranged from 41.29-94.27 μm, with an average of 63.45 μm and a CV of 16.14%, and the ploidies of 48.00% of the plants were greater than that of diploid, as shown in Table 4.

TABLE 4
Pollen size analysis of polyploid Iris domestica

| | | | | Coefficient of | High parental |
|---|---|---|---|---|---|
| Pollen | Diploid | Polyploid | Scope | variation | rate |
| Longitudinal | 60.16 | 65.83 | 44.30-98.22 | 18.36% | 54.00% |
| diameter (μm) | | | | | |
| Transverse | 57.32 | 63.45 | 41.29-94.27 | 16.14% | 48.00% |
| diameter (μm) | | | | | |

It is to be noted that when numerical ranges are recited in the claims of the present disclosure, it is to be understood that two endpoints of each numerical range and any numerical value between the two endpoints can be selected. In order to avoid redundancy, the present disclosure describes preferred examples.

While the preferred examples of the present disclosure have been described, additional variations and modifications to these examples can be made by those skilled in the art once the basic inventive concept is known. Therefore, the appended claims are intended to be interpreted as including the preferred example and all changes and modifications that fall within the scope of the present disclosure.

Obviously, those skilled in the art can make various modifications and variations to the present disclosure without departing from the spirit and scope of the present disclosure. Therefore, it is intended that the present disclosure cover the modifications and variations of the present disclosure provided that the modifications and variations are within the scope of the appended claims and equivalents.

1. A method for inducing Iris domestica seed buds to generate polyploid, comprising the following steps:
seed bud induction: inducing Iris domestica seeds at a low temperature, then soaking, sterilizing and germinating the same to obtain Iris domestica seed buds; and
polyploid induction: soaking the obtained Iris domestica seed buds in oryzalin solution with a mass fraction of 0.03-0.07 wt % for 18-36 h for polyploid induction to obtain mutagenized buds.

2. The method for inducing Iris domestica seed buds to generate polyploid according to claim 1, wherein a low induction temperature is 4-6° C. during the seed bud induction.

3. The method for inducing Iris domestica seed buds to generate polyploid according to claim 1, wherein a low temperature induction time is 30-40 d during the seed bud induction.

4. The method for inducing Iris domestica seed buds to generate polyploid according to claim 1, wherein the Iris domestica seeds are soaked with water for 30-36 h.

5. The method for inducing Iris domestica seed buds to generate polyploid according to claim 1, wherein 1.5%-2.0% sodium hypochlorite solution is used for soak and sterilization for 20-30 min during the seed bud induction.

6. The method for inducing Iris domestica seed buds to generate polyploid according to claim 1, wherein the germination is performed for 10-20 d.

7. The method for inducing Iris domestica seed buds to generate polyploid according to claim 1, wherein a length of the Iris domestica seed bud is 0.5-1 mm during the polyploid induction.

8. The method for inducing Iris domestica seed buds to generate polyploid according to claim 1, wherein the polyploid induction is performed by shaking during the polyploid induction.

9. The method for inducing Iris domestica seed buds to generate polyploid according to claim 1, further comprising the following steps: transplanting mutagenized seedlings, moisturizing the obtained mutagenized buds, and performing illumination culture, wherein the illumination culture conditions comprises a temperature of 23-25° C., a daytime illumination of 2000 lux for 14 h, and darkness at night for 10 h; and
transferring the seed buds into a nutrition pot after 2-3 cotyledons grow out, and putting the seedlings into a greenhouse for cultivation for 50-60 d after transplanting.

10. The method for inducing Iris domestica seed buds to generate polyploid according to claim 1, wherein the substrate of the nutrition pot is peat soil from Shandong mandelai agriculture technology co., ltd.
